(19) United States
(12) Patent Application Publication
(10) Pub. No.: US 2005/0032140 A1
(43) Pub. Date: Feb. 10, 2005

(54) Methods for predicting susceptibility to cardiovascular disease

(75) Inventors: Shinichiro Kurosawa, Edmond, OK (US); Deborah J. Stearns-Kurosawa, Edmond, OK (US)

Correspondence Address: FULBRIGHT & JAWORSKI L.L.P.;A REGISTERED LIMITED LIABILITY PARTNERSHIP, SUITE 2400, 600 CONGRESS AVENUE, AUSTIN, TX, 78701-3271, US

(73) Assignee: OKLAHOMA MEDICAL RESEARCH FOUNDATION

(21) Appl. No.: 10/868,748

(22) Filed: Jun. 15, 2004

Related U.S. Application Data
(60) Provisional application No. 60/483,386, filed on Jun. 27, 2003.

(51) Int. Cl. G01N 33/53; G01N 33/537; G01N 33/543

(52) U.S. Cl. 435/7.92

(57) ABSTRACT

The assay of soluble endothelial protein C receptor (sEPCR) is useful to predict cardiovascular disease, particularly atherosclerotic cardiovascular disease (ASCVD). An assay for sEPCR is therefore useful to identify individuals at risk of developing ASCVD. An sEPCR ELISA assay is particularly useful for this purpose. Elevated sEPCR is indicative of an increased risk of developing cardiovascular disease.

The present application claims benefit of priority to U.S. Provisional Application Ser. No. 60/483,386, filed Jun. 27, 2003, the entire contents of which are hereby incorporated by reference.

The government owns rights in the present invention pursuant to RO1 HL64787 AND RO1AI47575, both from the National Institute of Health.

### BACKGROUND OF THE INVENTION

I. Field of the Invention

The present invention relates to the fields of cardiology, vascular biology and inflammation. More particularly, it relates to assays to detect soluble endothelial protein C receptor (sEPCR) as a predictor of cardiovascular disease.

II. Related Art

The protein C pathway is a primary regulator of the coagulation system and the thrombotic events that contribute to atherosclerosis (Esmon et al., 1999). When circulating thrombin binds to thrombomodulin on the endothelial cell surface, it loses its ability to create clots. The bound thrombin instead activates the protein C zymogen to activated protein C (APC), which then inhibits critical cofactors (factors Va and VIIIa) of the clotting pathway. APC also enhances fibrinolysis by neutralizing PAI-1 (Sakata et al., 1986; Krishnamurti et al., 1991; de Fouw et al., 1993) and accelerating t-PA-dependent clot lysis. It is a series of events that occur on the endothelial surface and is subject to modulation by cytokines, serpin inhibitors, and products of neutrophil activation. The clinical importance of the protein C pathway is evidenced by the observation that homozygous protein C deficiency is life-threatening in neonates (Sanz-Rodriguez et al., 1999; Paret et al., 1998; Muller et al., 1996; Baliga et al, 1995); Dreyfus et al., 1995; Marlar et al., 1992; Petrini et al., 1990) and defects in this pathway account for more than half of the familial thrombophilia documented (Reitsma et al., 1995; De et al., 1998; Koster et al., 1993).

Multiple studies in non-human primates and in small clinical studies also demonstrate that the protein C pathway is critical to the host response to inflammation. In non-human primates, blockade of this system exacerbates the response to low level E. coli infusion, increasing both the inflammatory and coagulant responses (Taylor et al., 1987; Taylor, Jr., 2001; Gruber et al., 1990; Gruber et al., 1989). In patients with meningococcal septicemia, a devastating disease, treatment with protein C concentrates or activated protein C results in reversal of organ dysfunction accompanied by significantly reduced vascular complications and morbidity (Bernard et al., 2001; Weisel, 2002; Hodgson et al., 2002; Grinnell and Joyce, 2001; Betrosian et al., 1999; Clarke et al., 2000; Rivard et al., 1995). These observations in small studies have now been confirmed in a large-scale human study. In the recent PROWESS phase 3 clinical trial, patients with severe sepsis (n=1,690) were treated with APC (drotrecogin alpha, activated) (Bernard et al., 2001). APC reduced the 28-day all cause mortality rate by 19.4% in patients with severe sepsis.

The mechanism for APC's anti-inflammatory activity may include down-regulation of TNF-α generation by mononuclear cells (Murakami et al., 1997; Grey et al., 1994). Activated protein C also inhibits translocation of nuclear factor kB (NF-kB) in THP-1 monocytic cells treated with LPS (White et al., 2000). NF-kB is a critical transcription factor for LPS-induced pro-inflammatory cytokine production. APC may also regulate apoptosis by modulating expression of an endothelial Bcl-2 homolog, an inhibitor of apoptosis, and blocking induction of apoptosis by staurosporine Joyce et al., 2001). Additionally, in endothelial cells, APC initiates signaling events by binding to membrane-bound endothelial protein C receptor (EPCR, see below) and then proteolytically activating the tethered-ligand protease activated receptor 1 (PAR1) to initiate signaling (Riewald et al., 2002).

The endothelial protein C receptor (EPCR) is a recent member of the protein C pathway (Fukudome and Esmon, 1994). EPCR is a type 1 transmembrane protein specific to endothelium and is structurally homologous to CD1d/MHC class I proteins (Fukudome and Esmon, 1994; Oganesyan et al., 2002). The extracellular domain of EPCR binds both protein C and APC with similar affinities (Kd's˜30 nM) (Fukudome and Esmon, 1994; Liaw et al., 2000). Membrane-bound EPCR is important because on the endothelial surface, EPCR binds the protein C zymogen and presents it to the thrombin-thrombomodulin complex for augmented activation and propagation of the anticoagulant response (Stearns-Kurosawa et al., 1996). Blockade of EPCR in the primate model of sepsis, using an anti-EPCR monoclonal antibody (1494 mAb) that blocks ligand binding, resulted in early death and massive leukocyte infiltration, suggesting that EPCR influences leukocyte trafficking and responses to inflammatory challenge (Taylor et al., 2000).

In healthy adults, a soluble form of EPCR (sEPCR) exists in plasma (Kurosawa et al., 1997). Plasma sEPCR is the extracellular domain of the endothelial-bound parent and it circulates as a single, undegraded 40kDa molecule. sEPCR retains its ability to bind both protein C and activated protein C with affinity similar to the cell-bound receptor, but it no longer augments protein C activation (Kurosawa et al., 1997; Fukudome et al., 1996). In fact, high levels of sEPCR can inhibit protein C activation by binding protein C zymogen, effectively reducing the substrate concentration available for activation by thrombin/thrombomodulin (Fukudome et al, 1996). High levels of sEPCR also inhibit the anticoagulant activity of activated protein C (Fukudome et al., 1996; Regan et al., 1996) by binding activated protein C and altering its active site conformation and substrate recognition (Oganesyan et al., 2002; Liaw et al., 2000).

The current model for generation of sEPCR (FIG. 1) is that thrombin's interaction with its PAR-1 endothelial receptor stimulates up-regulation of surface metalloproteinase activity, which then cleaves membrane-bound EPCR to release the extracellular domain into the circulation (Xu et al., 2000). Thrombin does not directly cleave EPCR from the cell surface, but initiates the sEPCR shedding through interaction with PAR-1 because the agonist PAR-1 peptide was able to reproduce the response. The precise metalloproteinase responsible for cleaving membrane-bound EPCR from endothelial cells was not identified. Additional evidence demonstrating a role for thrombin in initiating sEPCR release comes from an in vivo study in rats (Gu et al., 2000). The data demonstrate increased EPCR mRNA and plasma sEPCR levels after stimulation with endotoxin and these effects were inhibited by hirudin, a specific thrombin inhibitor. Thus, sEPCR levels are regulated, probably as an indirect result of thrombin activity, and very high sEPCR levels in vitro will reduce APC anti-coagulant activity and limit the efficiency of the protein C pathway.

Several years ago, the present inventors developed a monoclonal antibody-based ELISA for measurement of sEPCR levels (U.S. Pat. No. 5,804,392) that is now available as a commercial kit (Asserachrom® sEPCR; Diagnostica Stago). They measured sEPCR levels in a variety of populations, both patients and asymptomatic healthy adults, in efforts to understand what sEPCR levels are reporting about the vasculature. They currently know five things about plasma sEPCR levels: (1) sEPCR levels have a bimodal distribution in apparently healthy adult populations (Steams-Kurosawa et al., 2003); (2) males have higher sEPCR levels than females (Steams-Kurosawa et al., 2003); (3) sEPCR levels increase in patients with systemic inflammatory diseases (Kurosawa et al., 1998; Boomsma et al, 2002); (4) sEPCR levels do not correlate with soluble thrombomodulin (sTM) levels, a marker of endothelial injury (Kurosawa et al., 1998; Boomsma et al., 2002); and (5) sEPCR levels decrease in response to treatment with heparin or oral anticoagulants (warfarin) (Stearns-Kurosawa et al., 2002).

The inventors also have measured sEPCR levels in several healthy adult populations from different countries and find an unusual bimodal distribution in each population (Steams-Kurosawa et al., 2003). sEPCR levels in about 80% of a population has a normal distribution with a mean of approximately 100-130 ng/ml, and males have higher sEPCR levels than females. In contrast, about 20% of each normal population had much higher sEPCR levels, up to 700+ ng/ml, that were not normally distributed (skewed).

The inventors have observed that plasma sEPCR levels are increased significantly in patients with sepsis, systemic lupus erythematosus or autoimmune vasculitis, suggesting a link with systemic inflammatory mechanisms (Kurosawa et al., 1998; Boomsma et al., 2002). Interestingly, in the patient groups for which we have studied both markers (sepsis, SLE, normal pregnancy, pre-eclampsia, angioplasty, type 2 diabetes, Wegener's granulomatosis), the plasma soluble EPCR and soluble thrombomodulin (sTM) levels did not correlate. Membrane-bound thrombomodulin is degraded on endothelial cells by neutrophil proteases (EPCR is not susceptible to neutrophil protease degradation) and multiple studies show that sTM levels are a good marker of endothelial injury (Takahashi et al., 1992; Takano et al., 1990; Wada et al., 1992). The inventors' consistent observation that sEPCR and sTM levels do not correlate raises the possibility that either they represent markers of endothelial injury from distinct vascular beds, or alternatively, that they are reporting different vascular conditions.

Based on their collective data, the inventors believe the latter explanation is more likely. Their model is that sTM levels report vascular damage whereas sEPCR levels report a vascular response to injury, particularly through thrombin activity. This concept is supported by their recent study demonstrating that plasma sEPCR levels decrease in response to heparin treatment in patients and to warfarin therapy in both normal volunteers and patients (Steams-Kurosawa et al., 2002). During warfarin administration, the sEPCR reduction paralleled decreases in F1+2 levels (the prothrombin fragment released during thrombin formation). When warfarin was discontinued, sEPCR levels returned almost to pre-treatment levels. Heparin and warfarin are anti-coagulants with very different mechanisms of action, but with the common end result of inhibiting thrombin formation. The observation that sEPCR generation is dependent on thrombin activity (in vitro at least) and that sEPCR levels decrease in response to anticoagulant treatment are consistent with the model that sEPCR levels report thrombin activity and an endothelial response to thrombin. Thus, it is possible that sEPCR levels are regulated and responsive to a pro-coagulant environment.

Several hemostatic and coagulation markers are used for the detection of a hypercoagulable state. Among them, the thrombin-antithrombin complex (TAT), prothrombin fragment 1+2 (F1+2) and fibrinopeptide A (FpA) measurements are considered to be representative. However, these markers have shortcomings in that they are not specific for in vivo generation of thrombin. They will report the generation of thrombin regardless of where thrombin was formed, including clot formation in a test tube or syringe while the blood was drawn. In contrast, sEPCR is formed from cleavage of membrane-bound EPCR expressed on the surface of endothelium in vivo. Thus, clot formation in a test tube cannot affect sEPCR levels. In fact, measurement of sEPCR levels in serum and plasma (citrate or heparin) correlate nicely.

It is increasingly evident that the vascular endothelium is an active participant in maintaining homeostasis. Recent insights into the basic mechanisms involved in atherogenesis indicate that deviation from normal endothelial physiology, or endothelial dysfunction, represents a key step in the development of atherosclerosis, plaque progression and atherothrombosis (Bonetti et al., 2003). However, relatively few markers are available to practicing physicians and cardiologists which report endothelial function or activation status.

Given its strategic location and biological properties, the endothelium serves as both a mechanical and biological barrier between the blood and the vascular wall. It also serves as an early and chronic target for molecular and cellular-mediated injury giving rise to plaque formation, vasoconstriction and progression to clinical manifestations. The risk to develop endothelial dysfunction may increase with the number of risk factors present in an individual (Vita et al., 1990) such that status of endothelial function is an integrated part of the overall cardiovascular risk burden (lifestyle, age, smoking, male, dyslipidemia, hypertension, obesity). Changes in endothelial function appear early and, if the changes are found to predict the clinical benefit of interventions, then drug and lifestyle changes may be evaluated more rapidly to reverse the disease progression (Celermajer, 1997). Thus, there remains a need in the art for more accurate measures of endothelial cell function.

### SUMMARY OF THE INVENTION

Thus, in accordance with the present invention, there is provided a method for predicting occurrence of an acute cardiovascular event in a subject comprising measuring circulating levels of soluble endothelial protein C receptor (sEPCR) in said subject, wherein elevated sEPCR levels, as compared to levels in normally distributed controls, predict occurrence of an acute cardiovascular event in said subject. sEPCR may be measured by an immunoassay, such as by ELISA. The sEPCR level may be determined by measuring sEPCR in a blood product, cerebrospinal fluid or urine, in particular in plasma or serum. The acute cardiovascular event may be selected from the group consisting of myocardial infarction, stroke, angina pectoris and sudden coronary death. In a particular embodiment, elevated sEPCR is greater than about 150 ng/ml serum. The subject may (a) not suffer from cardiovascular disease; (b) suffer from previously undiagnosed cardiovascular disease; or (c) suffer from previously diagnosed cardiovascular disease.

The method may further comprise assessing another cardiovascular disease risk factor in said subject, such as increased age, male gender, dyslipidemias (hypercholesteremia, hypertriglycaemia and low HDL-cholesterol), hypertension, C-reactive protein, hyperhomocysteinemia, lipoprotein(a), fibrinogen, obesity, physical inactivity, tobacco use, oral contraceptives, underlying primary disease (e.g., diabetes, SLE), and other hematological cardiovascular risk factors. The method may also further comprising administering a cardiovascular disease prevention or therapy program to said subject. The cardiovascular disease prevention program may be selected from the group consisting of diet and life style changes, low-dose aspirin, cholesterol reducing agents, blood pressure reducing agents, and oral anticoagulant treatment. The cardiovascular disease therapeutic program may be selected from the group consisting of beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists, statins, cytokine blockers/inhibitors and low fat diet. In addition, the method may further comprising assessing sEPCR after administering the cardiovascular disease prevention or therapy program to the subject.

In another embodiment, there is provided a method for predicting or diagnosing atherosclerotic cardiovascular disease in a subject comprising measuring circulating levels of soluble endothelial protein C receptor (sEPCR) in said subject, wherein elevated sEPCR levels, as compared to levels in normally distributed controls, predict development or diagnose presence of atherosclerotic cardiovascular disease in said subject. The sEPCR level may be determined by measuring sEPCR in a blood product, cerebrospinal fluid or urine, in particular in plasma or serum. In a particular embodiment, elevated sEPCR is greater than about 150 ng/ml serum. The subject may (a) not suffer from atherosclerotic cardiovascular disease or (b) have previously undiagnosed atherosclerotic cardiovascular disease.

The method may also further comprising administering an atherosclerotic cardiovascular disease prevention or therapy program to said subject. The atherosclerotic cardiovascular disease prevention program may be selected from the group consisting of diet and life style changes, low-dose aspirin, cholesterol reducing agents, blood pressure reducing agents, and oral anticoagulant treatment. The atherosclerotic cardiovascular disease therapeutic program may be selected from the group consisting of beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists, statins, cytokine blockers/inhibitors and low fat diet. In addition, the method may further comprising assessing sEPCR after administering the atherosclerotic cardiovascular disease prevention or therapy program to the subject.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Schematic diagram of sEPCR generation by thrombin and sEPCR function. A hypercoagulable, pro-inflammatory environment is characterized by increased thrombin generation in vivo. Thrombin upregulates metalloproteinase activity via PAR-1 on the endothelial cell surface. The metalloproteinase cleaves membrane-bound EPCR to release the extracellular domain, soluble EPCR (sEPCR), into the circulation. Membrane-bound EPCR accelerates the protein C activation rate by ˜5- to 10-fold. Because sEPCR binds protein C with similar affinity, it competes with membrane-bound EPCR for ligand, thereby inhibiting protein C activation efficiency. sEPCR also binds APC directly and inhibits the APC inactivation of coagulation cofactors Factor Va and Factor VIIIa, possibly contributing to phenotypic APC resistance. Thus, elevated sEPCR can influence both arms of the protein C pathway, by reducing the generation and activity of APC.

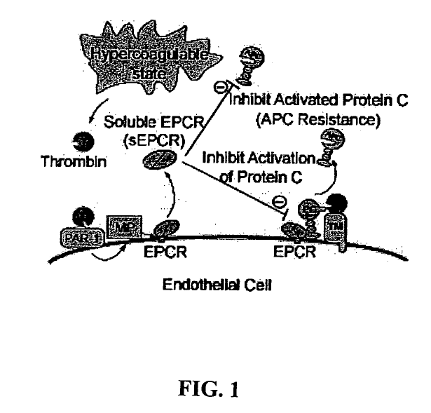

FIG. 2—Distribution of plasma sEPCR levels by gender and nationality. Plasma sEPCR levels in healthy donors from France and Italy. Values were grouped according to gender (horizontal) and nationality (vertical). The density traces (smoothed histograms) show the bimodal nature of each distribution.

FIG. 3—Box plots of the sub-population with lower sEPCR levels within each gender and nationality group. For each plot, the distance between ends of the lines represents the total range of the sample and the length of the box represents the range of the middle 50%. The patterns exhibited by the boxes indicate higher sample values for the Italians as compared to the French, and higher male values as compared to females. The means and standard deviations for each group are shown.

FIG. 4—Distribution of plasma sEPCR levels in Japanese healthy volunteers. Plasma sEPCR levels in healthy donors from Japan (n=141) were grouped and plotted by relative distribution. sEPCR levels show a bimodal distribution with the majority of individuals in the low sEPCR group (<200 ng/ml). The prevalence of subjects in Japan in the high sEPCR group (≧200 ng/ml) is only 9.9%, which is lower than the 21% observed in subjects from France.

FIG. 5—sEPCR levels are stable over time in healthy individuals. Plasma sEPCR levels in healthy donors were determined at the designated time points over a 48 hour period. There was little variation in their sEPCR levels, suggesting that simple changes in environment (e.g., diet) do not acutely affect sEPCR levels.

FIG. 6—Levels of soluble thrombomodulin (sTM) and soluble EPCR (sEPCR) in patients with sepsis or SLE. Plasma from patients with sepsis (n=16) or systemic lupus erythematosus (n=42) were assayed for the soluble receptors by ELISA. The means were compared by Student's t-test with receptor levels found in normal plasma (n=18).

FIG. 7—Plasma levels of sEPCR decrease in patients undergoing oral anticoagulant therapy. A French population of patients receiving oral anticoagulant therapy with acenocoumarol, fluindione, or warfarin had significantly lower sEPCR levels (right; 105.3±70.8 ng/ml; n=55) than a healthy French population (left; 165.8±115.8 ng/ml), p<0.0001.

FIGS. 8A-C—Levels of sEPCR respond to warfarin in healthy adult volunteers. Warfarin was administered to 6 adult volunteers for 8 days. The sEPCR levels (FIG. 8A), INR values (FIG. 8B) and F1+2 levels (FIG. 8C) were determined in blood drawn just before warfarin administration (day 0), throughout the administration period, and during a 1-week follow-up period. Data from each individual are indicated. The F1+2 levels are shown with the median, interquartile ranges, and 95% confidence intervals for the means.

FIGS. 9A-B—Levels of sEPCR decline in patients beginning warfarin treatment. Levels of sEPCR (FIG. 9A) and prothrombin fragment F1+2 (FIG. 9B) were determined in 7 patients before the start of oral anticoagulant therapy (time 0) and during treatment.

FIGS. 10A-B—Plasma sEPCR levels are higher in patients before angioplasty. The relative distribution of sEPCR levels determined in plasma from peripheral blood samples obtained from patients just prior to undergoing angioplasty (FIG. 10A; n=35) is significantly higher when compared with a healthy population (FIG. 10B; from France, n=200). ***The percentage of subjects in the high sEPCR group (≧200 ng/ml) is 60% in patients before angioplasty as compared with 21% in the healthy population (p<0.0001).

FIG. 11—Plasma sEPCR levels did not change in most patients immediately after angioplasty. sEPCR levels determined in plasma from peripheral blood samples obtained from patients just before angioplasty and after the procedure are compared. The linear regression analysis and 95% confidence limits are shown. In most patients, there was little change in their circulating sEPCR levels. This is consistent with the current model of sEPCR generation and we would not expect rapid changes in sEPCR levels via the thrombin-mediated mechanism. However, in two patients (circled), their sEPCR levels increased considerably. The re-occlusion rates is these patients is not known.

FIGS. 12A-C—High sEPCR levels decrease APC anticoagulant activity contributing to APC resistance. The APTT time was determined in normal plasmas (n=25)±added recombinant sEPCR (rsEPCR) without added APC (FIG. 12A) or with 60 nM APC (FIG. 12B). The APC sensitivity ratio (FIG. 12C) is the APTT+APC (s)/APTT (s). A lower APC sensitivity ratio is associated with APC resistance. Total sEPCR levels (endogenous plus recombinant) ranged from 677.7 ng/ml to 954.2 ng/ml (mean=745.7 ng/ml), which are at the upper ranges observed in clinical samples and healthy subjects. Data shown is mean ±S.E.M. The P values (paired t-test) are shown when p<0.05; n.s., not significant.

FIG. 13—Anti-EPCR blocking antibody reverses the effect of sEPCR. The anticoagulant activity of APC (1.3 nM) was determined with a one-stage Factor Xa clotting assay in the presence of 1.0 μM sEPCR with or without anti-EPCR monoclonal antibody which blocks binding of APC to EPCR. The blocking antibody neutralizes both exogenous and endogenous (plasma) sEPCR, thus further prolonging the clot times. Data shown is mean ±S.D. The P values (t-test) are shown.

### DETAILED DESCRIPTION OF THE INVENTION

Increasingly it is appreciated that cardiovascular disease, in particular atherosclerotic cardiovascular disease (ASCVD), is preceded by a prolonged hypercoagulable state, systemic vascular inflammation and endothelial dysfunction. The protein C pathway members, including the endothelial protein C receptor (EPCR), contribute to regulation of these coagulation and inflammatory events. Membrane-bound EPCR binds protein C, accelerates protein C activation and augments activated protein C anti-inflammatory activity. In a hypercoagulable environment, thrombin activity up-regulates metalloproteinases, which cleave the extracellular domain of EPCR, releasing it into the circulation. The resultant soluble EPCR (sEPCR) no longer contributes to protein C activation, but binds activated protein C and inhibits its anticoagulant function. This inhibition of the protein C pathway by elevated sEPCR levels shifts the equilibrium toward a hypercoagulable and pro-inflammatory state.

The inventors have found that about 20% of apparently healthy adults have significantly elevated sEPCR levels and that these levels decrease after oral anticoagulant therapy. These measurements of sEPCR levels in populations from several countries, as well as in selected patient cohorts, suggest that sEPCR is a marker for a hypercoagulable state that contributes to cardiovascular disease. Assays for measuring sEPCR have been described previously in U.S. Pat. No. 5,804,392 and U.S. Ser. No. 10/028,741. However, in this context, the assay was applied not as a predictor of disease in asymptomatic patients, but as a monitor of therapeutic efficacy in diseased patients.

### I. COAGULATION PATHWAY

The key component in the coagulation pathway is thrombin, which is the final enzyme product of the coagulation cascade and is necessary to form fibrin and to activate platelets, both of which are essential components of a clot. Since thrombin itself is virtually impossible to measure, use of surrogate markers is necessary. Currently, there are four tests used clinically to monitor thrombin generation: (a) prothrombin (PT) fragment 1+2 (F1+2); (b) fibrinopeptide A (FpA); (c) thrombin/antithrombin complexes (TAT); and (d) soluble fibrin monomer (SFM). Prothrombin fragment 1+2 is generated when prothrombin is converted to thrombin by factor Xa in the prothrombin complex. It is a peptide released into the circulation with a half-life of about 90 min. Therefore, F1+2 levels will reflect only acute thrombin generation.

Fibrinopeptide A (FpA) is released from fibrinogen by direct thrombin proteolysis. The peptide circulates in the blood and can be detected using a sensitive ELISA. The major drawback of FpA levels is they are very sensitive to in vitro generation of thrombin by phlebotomy techniques and blood handling routines. Some manufacturers have stopped making the ELISA kits and few hematology and coagulation reference laboratories offer the test.

Antithrombin (AT) is the primary inhibitor of thrombin and complexes with thrombin (TAT) to remove it from the circulation. TAT levels can be measured by ELISA. TAT levels also reflect acute generation of thrombin because the complexes are cleared by receptor-mediated mechanism in the liver, limiting evaluation of long-term thrombin levels. Importantly, both F1+2 and TAT levels will reflect both in vivo and artifactual in vitro thrombin generation. Thrombin can be generated in vitro, most commonly by phlebotomy procedures, artificially elevating F1+2 and TAT levels.

Soluble fibrin monomers (SFM) are generated when thrombin cleaves fibrinopeptides A and B from fibrinogen, and the fibrin monomers non-covalently interact with excess fibrinogen. Thrombin then generates factor XIIIa to cross-link the SFM into a local clot or systemic deposition on vascular walls. Thus, increased SFM is more an indication that the normal removal of thrombin is saturated or no longer working properly.

### II. sEPCR

EPCR is an endothelial cell receptor with significant homology to the CD1/MHC class 1 family (Fukudome and Esmon, 1994; Fukudome et al., 1996; Regan et al., 1996). The cloning and biological role of the endothelial cell receptor for protein C was described in PCT/US95/09636. The protein was predicted to consist of 238 amino acids, which includes a 15 amino acid signal sequence at the N-terminus, and a 23 amino acid transmembrane region which characterizes the receptor as a type 1 transmembrane protein. EPCR binds both protein C and APC with similar affinity (Fukudome et al., 1996) in the presence of calcium, and facilitates protein C activation by presenting the protein C substrate to the thrombin-thrombomodulin activation complex on cell surfaces (Steams-Kurosawa et al., 1996). Recombinant soluble EPCR (rsEPCR), truncated just before the transmembrane domain, binds both protein C and APC with an affinity similar to that observed for intact cell-surface expressed EPCR (Fukudome etal., 1996).

### III. IMMUNOASSAYS

It will be understood that polyclonal or monoclonal antibodies specific for the sEPCR will have utilities in several applications. These include the production of diagnostic kits and in methods of detecting sEPCR. Thus, the invention provides antibodies that bind immunologically to sEPCR. Means for preparing and characterizing antibodies are well known in the art (See, e.g., Antibodies: A Laboratory Manual, Cold Spring Harbor Laboratory, 1988; incorporated herein by reference). Antibodies to sEPCR have already been generated using such standard techniques and are described in U.S. Pat. No. 5,804,392 and U.S. Ser. No. 10/028,741.

A. Polyclonal Antisera

Polyclonal antisera is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, or the animal can be used to generate mAbs (below).

For production of rabbit polyclonal antibodies, the animal can be bled through an ear vein or alternatively by cardiac puncture. The procured blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix or protein A followed by antigen (peptide) affinity column for purification.

B. Monoclonal Antibodies

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified sEPCR protein, polypeptide or peptide. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like. The selected hybridomas would then be serially diluted and cloned into individual antibody-producing cell lines, which clones can then be propagated indefinitely to provide mAbs. The cell lines may be exploited for mAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide mAbs in high concentration.

The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

C. Immunoassays

In still further embodiments, the present invention thus concerns immunodetection methods for binding, quantifying or otherwise generally detecting sEPCR. The steps of various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), radioimmunoassays (RIA) and immunobead capture assay. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, one would obtain a sample suspected of containing sEPCR, and contact the sample with an antibody, and then detect or quantify the amount of immune complexes formed under the specific conditions.

Contacting the chosen biological sample with the antibody or antisera under conditions effective and for a period of time sufficient to allow the formation of immune complexes (primary immune complexes) is generally a matter of simply adding the composition to the sample and incubating the mixture for a period of time long enough for the antibodies to form immune complexes with sEPCR. After this time, the sample-antibody composition will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference.

In certain embodiments, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

Of particular interest in the present invention are enzyme linked immunosorbent assays, known as ELISAs. In one exemplary ELISA, antibodies binding to the encoded proteins of the invention are immobilized onto a selected surface exhibiting protein affinity, such as a well in a polystyrene microtiter plate. Then, a test composition suspected of containing the sEPCR is added to the wells. After binding and washing to remove non-specifically bound immunocomplexes, the bound antigen may be detected.

Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the sEPCR are immobilized onto the well surface and then contacted with the antibodies of the invention. After binding and washing to remove non-specifically bound immune complexes, the bound antibody is detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the sEPCR are immobilized, involves the use of antibody competition in the detection. In this ELISA, labeled antibodies are added to the wells, allowed to bind to the sEPCR and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. For example, in coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is more customary to use a secondary or tertiary detection means rather than a direct procedure. Thus, after binding of a protein or antibody to the well, coating with a non-reactive material to reduce background, and washing to remove unbound material, the immobilizing surface is contacted with a control and sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand. “Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hrs, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hrs at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-3-ethyl-benzthiazoline-6-sulfonic acid (ABTS) and H₂O₂, in the case of peroxidase as the enzyme label. Quantitation is then achieved by measuring the degree of color generation, e.g., using a visible spectra spectrophotometer.

D. sEPCR Levels

sEPCR cut off levels are not uniform. As discussed in the Examples, one should perform standard evaluations of patient populations to establish appropriate cut off levels based on normally distributed subjects. For example, in the French population, the cut point and proportion higher was 155 ng/ml and 22% for females, 176 ng/ml and 25% for males. For the Italian population, the cut point and proportion higher was 190 ng/ml and 24% for females, 200 ng/ml and 25% for males. In Japanese adults, the inventors again observed a bimodal distribution of sEPCR levels. The frequency of those with high sEPCR levels (≧200 ng/ml) was 9.9%. This was substantially less than that observed in the French and Italian populations (20-25%), suggesting there may be a genetic component contributing to elevated sEPCR levels. However, the bimodal distribution is still observed.

Thus, in each large population of apparently healthy adults that have been studied, sEPCR levels in the majority of subjects are “normally distributed” (Gaussian distribution), usually averaging about 100-150 ng/ml. This sub-population is referred to as “normal distributed controls” for the purpose of this application to distinguish from the sub-population with high sEPCR levels that are not normally distributed (non-Gaussian distribution) and can range up to 700 ng/ml or higher. Thus, in general terms, about 80% of an apparently healthy adult population will have sEPCR levels<200 ng/ml (low sEPCR group) and these values are normally distributed (Gaussian distribution). The remaining 20% of the population has much higher levels, ranging up to 700+ng/ml sEPCR (high sEPCR group, >200 ng/ml).

### IV. CARDIOVASCULAR DISEASE: DIAGNOSIS, PREVENTION AND THERAPY

Cardiovascular disease is one of the leading causes of morbidity and mortality in the world. In the U.S. alone, estimates indicate that over 60 million people are currently living with with some form of the disease—high blood pressure, congestive heart failure, coronary heart disease (atherosclerosis, chronic ischemic heart disease, myocardial infarction, angina pectoralis), cardiomyopathy (cardiac hypertrophy, dilated cardiomyopathy) and stroke—and claims upwards of one million lives each year. The following is a general discussion of various cardiovascular disease states, their diagnosis, prevention and therapy.

A. Cardiovascular Disease Risk Factors

In conjunction with the present invention, it may prove useful to combine other diagnostic procedures with the sEPCR assays of the present invention. The following is a discussion of a various risk factors and diagnostic/prognostic factors for use in conjunction with the present invention.

1. Coronary Heart Disease (CHD)

Typical risk factors for coronary heart disease (CHD), and hence the basis for diagnosis, are high blood pressure, high blood cholesterol and diabetes. An electrocardiogram, along with a patient interview and exam, will be performed. Other tests may be ordered such as stress testing, cardiac ultrasound, cardiac catheterization, or nuclear imaging.

2. Congestive Heart Failure (CHF)

Because congestive heart failure (CHF) typically results from other cardiac diseases, most commonly, asymptomatic patients are not usually screened for CHF. When patients present symptoms that may indicate CHF, diagnostic tests can be performed to determine the cause. In some cases, results from coronary artery disease screening methods (blood cholesterol tests, blood pressure measurements, stress tests, etc.) may also indicate CHF and warrant further examination.

Early symptoms of CHF may include fatigue, dyspnea (shortness of breath), and wheezing or hoarseness. Other noticeable symptoms include heart palpitations, swollen ankles and legs, and weight gain (due to fluid build-up). Depending on the extent of CHF, patients may also have other complications including an abnormal heart murmur, pulmonary congestion arrhythmia, (irregular heartbeat), hypertrophy (enlarged heart), tachycardia (accelerated heartbeat), and kidney or liver problems.

Diagnosing CHF typically begins with a physical exam, including careful documentation of the patient's medical and personal history of heart problems (i.e., atherosclerosis—narrowing of arteries, coronary artery disease, history of CHF or congenital heart disease).

3. Cardiomyopathy

Diagnosis of dilated cardiomyopathy and/or cardiac hypertrophy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

B. Prophylaxis

A few prophylactic measures may be taken to reduce the risk of acute cardiovascular events, as well as more chronic forms of cardiovascular disease. Such preventative protocols include exercise, reduced fat diets, low-dose aspirin, statins and oral anticoagulants.

C. Therapies

A variety of “standard” pharmaceutical cardiac therapies exits. Examples of standard therapies include, without limitation, so-called “beta blockers,” anti-hypertensives, cardiotonics, anti-thrombotics, vasodilators, hormone antagonists, ionotropes, diuretics, endothelin antagonists, calcium channel blockers, phosphodiesterase inhibitors, ACE inhibitors, angiotensin type 2 antagonists and cytokine blockers/inhibitors.

In addition, combinations of the foregoing drugs may be used by administration of a single composition or pharmacological formulation that includes multiple agents, or by administering two distinct compositions or formulations, at the same time. Alternatively, one drug may precede or follow administration of the other by intervals ranging from minutes to weeks. In embodiments where the two agents are applied separately, one would generally ensure that a significant period of time did not expire between the time of each delivery, such that both agents would still be able to exert an advantageously combined effect. In such instances, it is contemplated that one would typically administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. It also is conceivable that more than one administration of a drug will be desired.

By way of illustration, the following permutations based on 3 and 4 total administrations are exemplary, where A represents a first drug and B represents a second drug:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

D. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Distribution of sEPCR Levels in Healthy Adults is Skewed and Significantly Different from a Normal Distribution

The distribution of sEPCR levels in healthy adults was evaluated using plasma samples from healthy adult populations obtained from collaborators in France and Italy. All samples in this and consecutive studies were collected with informed consent using IRB-approved protocols. sEPCR levels were measured by a monoclonal antibody-based ELISA (Stearns-Kurosawa et al., 2002) adapted from a prior method (Kurosawa et al., 1998). Samples were grouped by gender and nationality (Italian males, Italian females, French males and French females). sEPCR values in these four gender by nationality groups were tested for normality using the Shapiro-Wilk test. Since all showed significant departure from normality, the Kruskal-Wallis test was used to examine differences among the four populations. Since histograms suggested subpopulations within each group, each group was partitioned into low and high sEPCR populations by first ordering all values from smallest to largest. Next, the point where the two populations were apparently separated by at least ten units was used as an initial partition point. If the resulting lower population contained a high outlier (as defined by box plot criterion), the partition point was lowered by one value until no outlier was observed. The resultant populations were tested for normality. The four lower populations were deemed suitable for a 2×2 (gender by nationality) analysis of variance. The four higher populations were deemed not suitable for parametric analysis and were examined using the Kruskal-Wallis test, a nonparametric analog of analysis of variance.

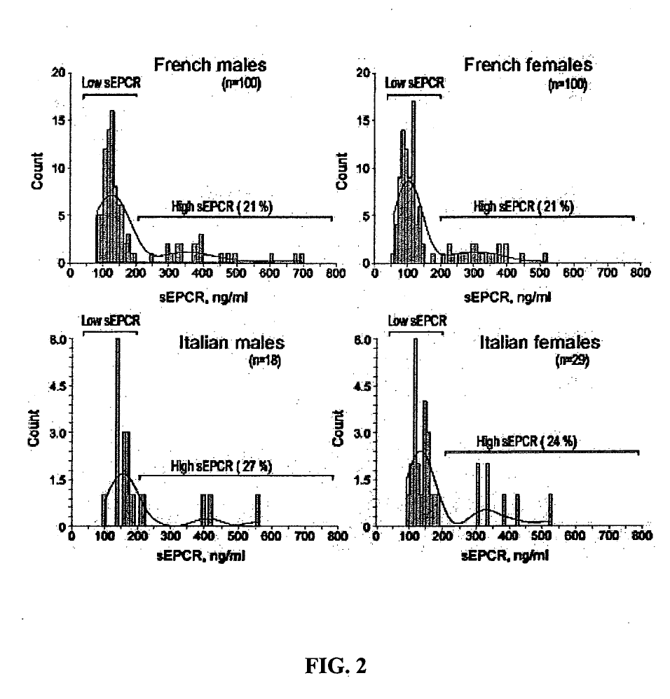

The analysis of plasma sEPCR levels in the four groups (males and females from Italy or France) of apparently healthy adults demonstrated that each was significantly different from a normal distribution (Steams-Kurosawa et al., 2003). Each group had a bimodal distribution of plasma sEPCR values (FIG. 2) and the Kruskal-Wallis test indicated a significant difference (p<0.0001) among the four groups. The partitioning algorithm yielded different cut points for high/low division for each group, but surprisingly similar proportions in the high sEPCR groups. For the French population, the cut point and proportion higher was 155 ng/ml and 22% for females, 176 ng/ml and 25% for males. For the Italian population, the cut point and proportion higher was 190 ng/ml and 24% for females, 200 ng/ml and 25% for males.

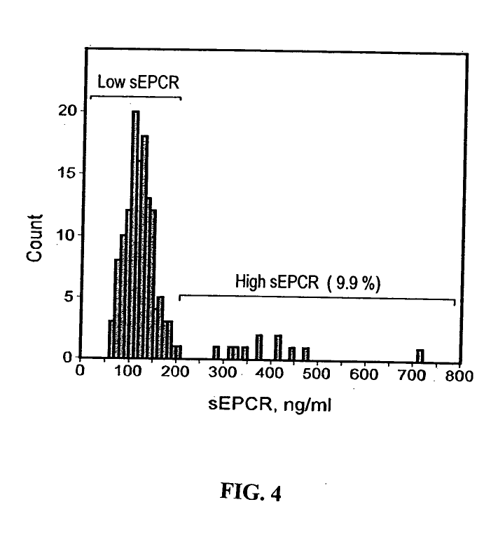

For purposes of reporting data in the examples set forth below, the cut point for all adult populations was set at 200 ng/ml based on visual inspection of the data (FIGS. 2 and 4). Those in the low sEPCR group have levels <200 ng/ml; those in the high sEPCR group have levels ≧200 ng/ml. This cut point probably underestimates the percentage of individuals in the high sEPCR group, but is useful for general comparison of large populations. Data for all populations should undergo rigorous analysis to determine cut points for statistical comparisons, as was done with the French and Italian samples. This may be performed by one of ordinary skill in the art using the techniques described herein.

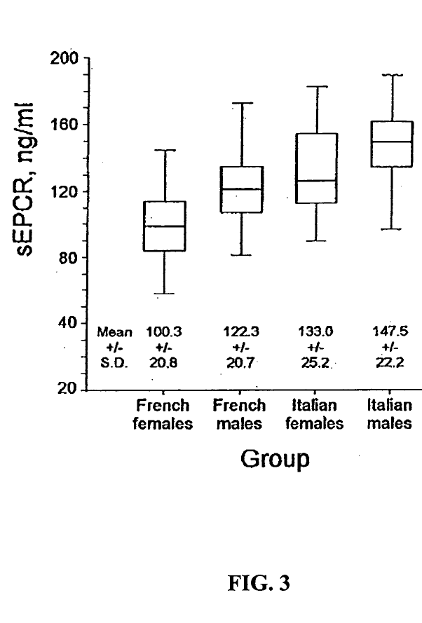

The low sEPCR groups for each of the four gender and nationality populations (FIG. 2) showed a normal distribution (Gaussian) and were examined using a two factor analysis of variance. The means and standard deviations are indicated (FIG. 3). Both gender and nationality main effects were significant (p<0.0001), but there was no significant interaction between gender and nationality (p=0.35). In other words, males had higher sEPCR levels, but this gender difference was similar regardless of the country of origin. In these groups, the estimated difference in sEPCR levels between the two nationalities was 28.9 ng/ml, and the gender difference was 18.2 ng/ml sEPCR. In each large population of apparently healthy adults that have been studied, sEPCR levels in the majority of subjects are normally distributed (Gaussian distribution), usually averaging about 100-150 ng/ml. It is this sub-population that is referred to as “normal controls,” to distinguish from the sub-population with high sEPCR levels that are not normally distributed (non-Gaussian distribution) and can range up to 700 ng/ml or higher.

The inventors have also measured sEPCR levels in a population of apparently healthy Japanese adults. These samples were obtained from Dr. Hideo Wada (Dept of Clinical Laboratory Medicine, Mie University School of Medicine, Japan). The inventors were particularly interested in sEPCR levels in an Asian population because they have a lower incidence of ASCVD and the genetic contribution to thrombophilia is very different from western populations. For example, unlike a western population, Asians do not have a high incidence of Factor V Leiden (De et al., 1998; Ozawa et al., 1996; Lee, 2001; Pepe et al., 1997). This mutation in Factor V (R506Q) renders it resistant to cleavage by activated protein C, severely diminishing the effectiveness of protein C anti-coagulant mechanisms (Dahlback et al., 1997). Factor V Leiden is the most prevalent genetic thrombophilia in individuals of European descent, significantly contributing to risk of thrombotic events, particularly in homozygous individuals and those with coinheritance of other risk factors.

In the Japanese adults, the inventors again observed a bimodal distribution of sEPCR levels (FIG. 4). The frequency of those with high sEPCR levels (≧200 ng/ml) was 9.9%. This was substantially less than that observed in the French and Italian populations (20-25%), suggesting there may be a genetic component contributing to elevated sEPCR levels. However, the bimodal distribution is still observed.

Thus, in general terms, about 80% of an apparently healthy adult population will have sEPCR levels<200 ng/ml (low sEPCR group) and these values are normally distributed (Gaussian distribution). The remaining 20% of the population has much higher levels, ranging up to 700+ng/ml sEPCR (high sEPCR group, ≧200 ng/ml). In the Japanese population, the frequency of those with high sEPCR levels was less (9.9%), but present nevertheless.

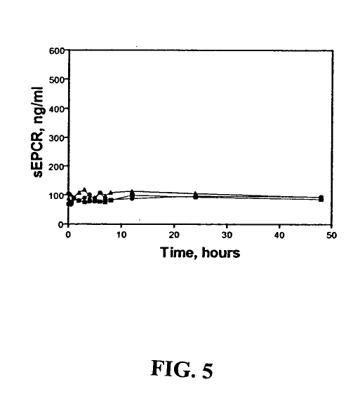

To show that this is not just an artifact of sampling bias, the inventors also found that sEPCR levels do not change much with time in a given individual, at least over the course of 2 days (FIG. 5). This suggests that simple daily changes, such as diet (short-term) or biorhythms (e.g., cortisol), do not acutely influence sEPCR levels.

### Example 2

### sEPCR Levels are Increased in Patients with Systemic Inflammatory Diseases and do not Correlate with Soluble Thrombomodulin (sTM) Levels

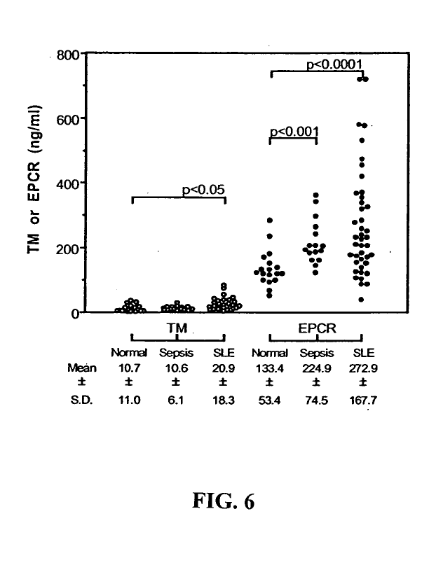

Previously, the inventors showed that sEPCR levels are elevated in patients with sepsis or systemic lupus erythematosus (SLE) (Kurosawa et al., 1998). Both are systemic diseases with prominent coagulopathic and inflammatory components. SLE in particular promotes premature coronary atherosclerosis and cerebrovascular disease, and SLE patients experience a greatly increased risk of coronary events compared to age and sex matched controls (Urowitz et al., 1976; Petri, 2000). The septic patients required hemodynamic support, had ATIII levels less than 70%, no previous liver disease and no hematologic disease. In both patient populations, sEPCR levels were significantly increased (FIG. 6). They did not observe a correlation between the sEPCR levels and multiple organ failure score, survival or presence of septic shock. Surprisingly, parallel measurements of soluble thrombomodulin (sTM) did not show any correlation with the sEPCR levels in either the SLE patients (r²=0.018) or in the septic patients (r²=0.013). This was surprising because both TM and EPCR are located primarily on the endothelium. However, membrane-bound EPCR is expressed almost exclusively on large vessels along with TM, but it is not nearly as abundant as TM in the microvasculature (Laszik et al., 1997). These studies suggest that sEPCR levels may reflect large vessel disease processes (a prominent feature of atherosclerosis), and also raised the possibility that sEPCR levels may arise from mechanisms distinct from neutrophil-mediated protease activity that give rise to circulating soluble TM.

### Example 3

### sEPCR Levels are Linked to Thrombin Production in Vivo

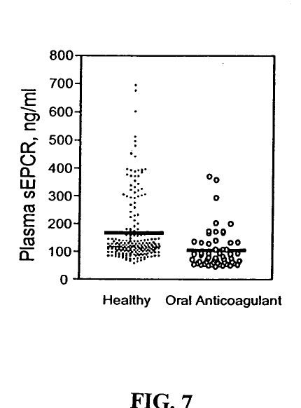

Earlier in vitro studies indicated that generation of sEPCR is regulated by inflammatory mediators (Gu et al., 2000), including thrombin-mediated up-regulation of surface metalloproteolytic activity (Xu et al., 2000). Therefore, the inventors addressed the question of whether plasma sEPCR levels reflect changes in thrombin levels in vivo (Stearns-Kurosawa et al., 2002). They found that sEPCR levels were reduced significantly in French patients undergoing oral anticoagulant therapy with vitamin K antagonists, including acenocoumarol, fluindione or warfarin (p<0.0001; FIG. 7). The length of treatment time was not available nor did we have gender or other demographic information for further analysis of this population.

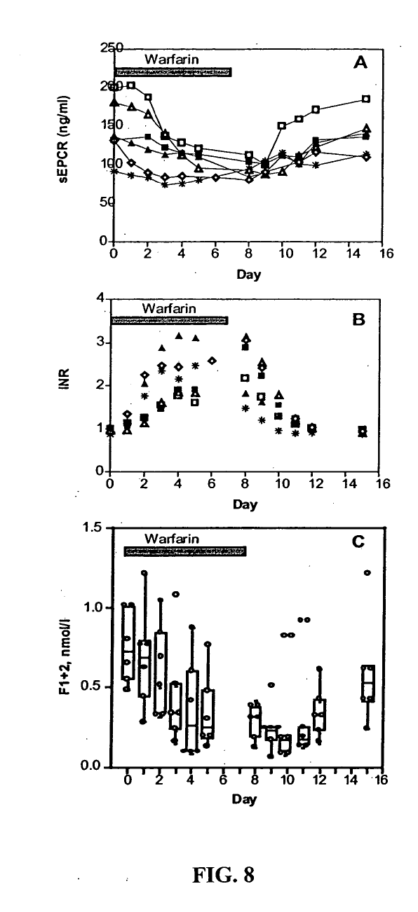

Therefore, in a small prospective study, the inventors evaluated the influence of oral anticoagulant treatment on sEPCR levels in normal adult volunteers. sEPCR levels declined to ˜100 ng/ml within 2-3 days upon initiation of an 8 day warfarin anticoagulant treatment period and subsequently increased within 2-3 days after cessation of warfarin (FIG. 8A). Plasma sEPCR levels returned to pre-treatment values within 1 week. Changes in their plasma sEPCR levels mirrored changes in their INR values (FIG. 8B) and decreases in F1+2 levels (FIG. 8C) during warfarin treatment. The INR is a standardized clotting time assay (should be prolonged with warfarin) and F1+2 is a fragment released from prothrombin during thrombin generation and is assayed with an ELISA.

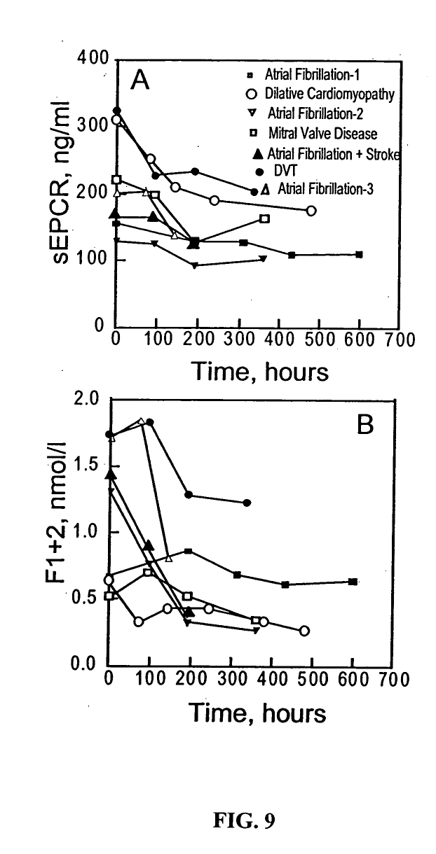

A similar decline in sEPCR levels over time was observed in seven patients initiating warfarin treatment for thrombotic disorders, including deep vein thrombosis, atrial fibrillation and dilative cardiomyopathy (FIG. 9A). F1+2 levels also decreased in the warfarin-treated patients (FIG. 9B). These studies demonstrate that plasma levels of sEPCR decline in response to treatment with anticoagulants whose mechanism of action is known to decrease in vivo thrombin production. These studies demonstrate that sEPCR levels are responsive to anticoagulant therapy, providing an important link between in vivo thrombin production during disease and subsequent endothelial responses.

### Example 4

### sEPCR Levels are Increased in Patients Targeted for Coronary Angioplasty

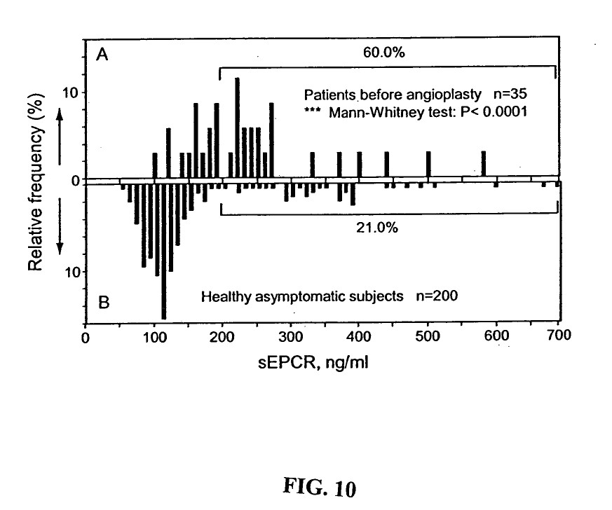

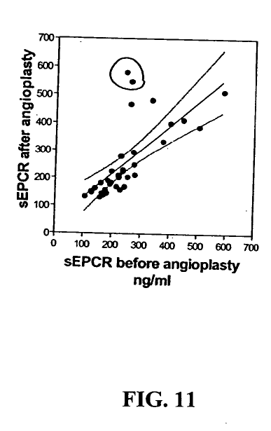

Coronary artery disease is a major complication of the atherosclerotic process and we evaluated sEPCR levels in a cohort of thirty-five patients undergoing coronary angioplasty (PTCA). These samples were obtained from a collaborator at Washington University School of Medicine. Peripheral blood samples were drawn both before and after the angioplasty procedure. The inventors observed that sEPCR levels were increased in the patients before angioplasty (FIG. 10A) when compared to a healthy population (FIG. 10B; samples from France). The percentage of individuals with sEPCR levels ≧200 ng/ml was 60% (21/35) in the patients before angioplasty and 21% in the healthy population (42/200; from France). Plasma sEPCR levels in samples obtained from the coronary sinus did not differ from the peripheral blood results (not shown). The inventors further observed that in most of the patients, the sEPCR levels did not change after the PTCA procedure (FIG. 11). This is consistent with the understanding of how sEPCR is generated (indirectly via thrombin-mediated up-regulation of metalloproteinase activity), so that one would not expect to see immediate changes in sEPCR levels. Of interest, though, are 2 patients whose sEPCR levels increased ˜2-fold after the angioplasty. The inventors speculate that this increase in sEPCR is due to endothelial/vascular damage incurred as a result of the surgical intervention; most likely their sTM levels also increased after the procedure, although this was not measured. The re-occlusion rates in these patients is not known. This study demonstrates that individuals with coronary artery disease, most likely the consequence of advanced atherosclerosis, have elevated sEPCR levels relative to a healthy population. The relative distribution of sEPCR levels in the angioplasty patients remained bimodal, with some patients having much higher sEPCR levels compared to the majority.

### Example 5

### High sEPCR Levels may Contribute to APC Resistance

Activated protein C (APC) is an anti-coagulant enzyme responsible for inhibition of thrombin formation by virtue of its proteolytic inactivation of coagulation cofactors Va and VIIIa. A reduced anticoagulant response to APC, known as APC resistance, is found in 10-50% of patients with venous thrombosis (Koster et al., 1993; Griffin et al., 1993; Rodeghiero and Tosetto, 1999). APC resistance due to the presence of the Factor V Leiden polymorphism (Arg506Gln) is inherited as an autosomal dominant trait with a prevalence of 2-13% in the general population. The factor V Leiden molecule has normal pro-coagulant activity when activated by thrombin or Factor Xa, but the rate of inactivation by APC is about 10-fold slower than normal Va because the mutation is in a cleavage site recognized by APC. The Factor V Leiden polymorphism renders the molecule resistant to APC proteolysis, thereby contributing to APC resistance and up to an 80-fold increase in risk of thrombosis in individuals with a homozygous defect (Rosendaal et al., 1995). This defect is most common is individuals of European descent and virtually absent in Asian populations (De et al., 1998; Ozawa et al., 1996; Lee, 2001; Pepe etal., 1997).

Interestingly, in those with normal Factor V, phenotypic APC resistance is found in about 15% of the population (Rodeghiero and Tosetto, 1999) and is associated with increased risk of ischemic stroke (van der Bom et al, 1996). Thus, while it is clear that homozygous Factor V Leiden is a thrombotic risk factor, it is becoming increasingly apparent that other pathways also may contribute to a poor anticoagulant response to APC (Taube et al., 1999). These include oral contraceptive use (Rosing et aL, 1999), autoantibodies against APC (Zivelin et al, 1999) or Factor V (Kalafatis et al., 2002), as well as availability of glucosylceramide (Deguchi et al., 2002; Deguchi et al., 2001) or high density lipoprotein (Griffin et al., 1999; Griffin et al., 2001).

In in vitro clotting assays, recombinant sEPCR reduces the anticoagulant response to APC (Regan et al., 1996), raising the possibility that plasma sEPCR may contribute to APC resistance. However, these studies were done with very large molar excesses of sEPCR (>500 nM or >21,000 ng/ml), greatly exceeding levels found in apparently healthy donors or in patients (see FIGS. 2-4, 6 and 10). In some individuals in the healthy populations, sEPCR levels ranged up to 700 ng/ml (high sEPCR group), and some in the patient populations reached 800-900 ng/ml.

The inventors undertook a preliminary study to address the possibility that high sEPCR levels within observed pathophysiological ranges affect APC anticoagulant activity, possibly contributing to APC resistance. This study was done in collaboration with Dr. Nicola Englyst (Endocrinology & Metabolism Unit, Southampton University Hospitals Trust, Southampton, UK). APC resistance was measured using Coatest APC Resistance-C kits (Chromogenix via Quadratech, UK). This is an APTT-based clotting assay. The time taken for a clot to form was measured in the absence and presence of added APC. The APC sensitivity ratio was calculated as the APTT time in the presence of APC (seconds) divided by the APTT time (seconds). A lower APC sensitivity ratio is associated with APC resistance. All assays were carried out at least in duplicate. Data were normalized by log transformation and were analyzed using paired Students t tests. Kit-to-kit variation was normalized against the data obtained using characterized control samples (n>10).

Plasma from 25 young, healthy adult males was used in this study. Because the inventors knew that the majority of samples would be in the low sEPCR group, they chose to add recombinant sEPCR (the extracellular ligand-binding domain of EPCR (Fukudome et al., 1996; Regan et al., 1996) to 600 ng/ml to each sample so that they would have sEPCR levels in the 700-900 ng/ml range (based on FIGS. 2 and 6). In the current study, the plasma sEPCR levels before the addition of recombinant sEPCR ranged from 77.3 ng/ml-354.2 ng/ml (mean ±S.D.=145.7±76.2 ng/ml). Thus, total sEPCR levels (endogenous plus recombinant) ranged from 677.7 ng/ml-954.2 ng/ml (mean=745.7 ng/ml), which are at the upper ranges observed in clinical samples and healthy subjects.

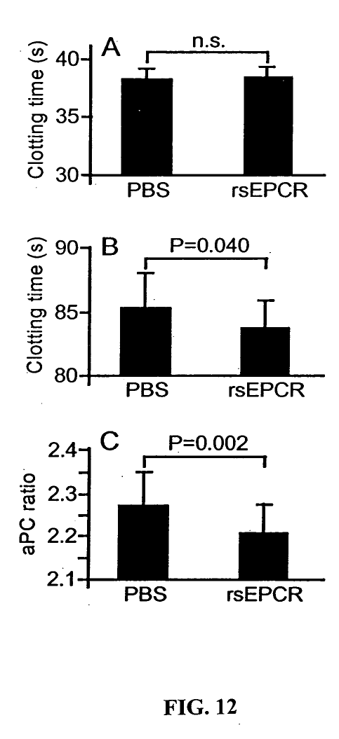

In the absence of APC, the added recombinant sEPCR had no effect on clotting times (FIG. 12A). As shown in FIG. 12B, addition of recombinant sEPCR to 600 ng/ml and APC to the samples resulted in decreased APC activity. Correspondingly, the APC sensitivity ratio decreased (FIG. 12C; 2.27±0.08, PBS control vs. 2.21±0.07, rsEPCR, p=0.002). It should be noted that the error bars shown reflect the variability of the clotting time data between different individuals and do not indicate inherent variability in the effect of recombinant sEPCR between individuals.

Example 6

### Blocking Plasma sEPCR with an Anti-EPCR Antibody Increases the Anticoagulant Activity of APC

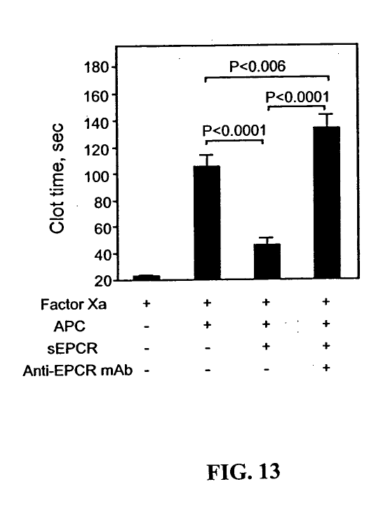

To directly examine the influence of endogenous sEPCR in plasma, an anti-EPCR monoclonal antibody (1494 mAb; IgG1ₖ), which blocks both protein C and APC binding, was added to neutralize sEPCR in plasma. Clotting times were determined with a one-stage factor Xa clotting assay on pooled human plasma (25 donors) in the presence of APC, recombinant sEPCR and/or excess anti-EPCR mAb (83 μg/ml final) (FIG. 13). As expected, addition of APC prolonged the clotting times and addition of recombinant sEPCR inhibited the APC activity. When the blocking anti-EPCR mAb was included in molar excess, inhibition by the sEPCR was reversed and the APC activity increased. The prolongation actually was greater than that observed without recombinant sEPCR (p<0.006), indicating that the anti-EPCR antibody blocked both exogenous and endogenous sEPCR. Thus, blocking endogenous plasma sEPCR with the antibody resulted in greater APC anti-coagulant activity.

Taken together, these observations support the notion that pathophysiological levels of sEPCR can reduce APC anti-coagulant activity. This may contribute to APC resistance and may be an additional pro-coagulant burden, particularly in patients with both compromised protein C levels (e.g., sepsis) and high sEPCR levels.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the method described herein, without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference:
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 5,804,392
U.S. Ser. No. 10/028,741
Antibodies: A Laboratory Manual, Cold Spring Harbor Laboratory, Cold Spring Harbor Press, Cold Spring Harbor, New York, 1988.
Baliga et al., Eur. J. Pediatr., 154:534-538, 1995.
Bernard et al, N. Engl. J., Med., 344:699-709, 2001.
Bernard et al., Crit. Care Med., 29:2051-2059, 2001.
Betrosian et al., Crit. Care Med., 27:2849-2850, 1999.
Bonetti et al, Arterioscler. Thromb. Vasc. Biol., 23:168-175, 2003.
Boomsma et al., Clin. Exp. Immunol., 128:187-194, 2002.
Buffon et al., N. Engl. J. Med., 347:5-12, 2002.
Campbell, In: Monoclonal Antibody Technology, Laboratory Techniques in Biochemistry and Molecular Biology, Burden and Von Knippenberg (Eds.), Elseview, Amsterdam, 13:71-74; 75-83, 1984.
Celermajer, J. Am. Coll. Cardiol., 30:325-333, 1997.
Clarke et al., Intensive Care Med., 26:471-473, 2000.
Cooke, Proc. Natl. Acad. Sci. USA, 100:768-770, 2003.
Dahlback et al., Haemostasis, 26(Suppl)4:301-314, 1996.
De et al., Semin. Thromb. Hemost., 24:367-379, 1998.
de Fouw et al., Blood Coagul. Fibrinolysis, 4:201-210, 1993.
Deguchi et al., Blood, 97:1907-1914, 2001.
Deguchi et al., J. Biol. Chem., 277:8861-8865, 2002.
Dreyfus et al., Semin. Thromb. Hemost., 21:371-381, 1995.
Esmon et al., Haematologica, 84:363-368, 1999.
Esmon, In: Handbook of Experimental Pharmacology, Born et al., (Eds.), Springer-Verlag, NY, 447-476, 1999.
Fukudome and Esmon, J. Biol. Chem., 269:26486-26491, 1994.
Fukudome et al., J. Biol. Chem., 271(29):17491-17498, 1996.
Fukudome and Esmon, J. Biol. Chem., 269:26486-26491, 1994.
Galis et al., J. Clin. Invest., 94:2493-2503, 1994.
Gefter et al., Somatic Cell Genet., 3(2):231-236, 1977.
Goding, In: Monoclonal Antibodies: Principles and Practice, 2d ed., Academic Press, Orlando, Fla., 60-66, and 71-74, 1986.
Grey et al., J. Immunol., 153:3664-3672, 1994.
Griffin et al., Blood, 82:1989-1993, 1993.
Griffin et al., J. Clin. Invest., 103:219-227, 1999.
Griffin et al., Thromb. Haemost., 86:386-394, 2001.
Grinnell and Joyce, Crit. Care Med., 29:S53-S60, 2001.
Gruber et al., Blood, 73:639-642, 1989.
Gruber et al., Circulation, 82:578-585, 1990.
Gu et al, Blood, 95:1687-1693, 2000.
Hodgson et al., Br. J Haematol., 116:905-908, 2002.
Jialal and Devaraj, Am. J Clin. Pathol., 116(Suppl):S108-S115, 2001.
Joyce et al., J. Biol. Chem., 276:11199-11203, 2001.
Kai et al., J. Am. Coll. Cardiol., 32:368-372, 1998.
Kalafatis et al., Blood, 99:3985-3992, 2002.
Kohler and Milstein, Nature, 256(5517):495-497, 1975.
Kohler and Milstein, Eur. J. Immunol., 6(7):511-519, 1976.
Koster et al., Lancet., 342:1503-1506, 1993.
Kovanen et al., Circulation, 92:1084-1088, 1995.
Krishnamurti et al., J. Lab. Clin. Med., 118:523-530, 1991.
Kurosawa et al., J. Clin. Invest., 100:411-418, 1997.
Kurosawa et al., Blood, 91:725-727, 1998.
Laszik et al., Circulation, 96:3633-3640, 1997.
Laszik et al., Am. J. Pathol., 159:797-802.
Lee, Am. J. Med. Sci., 322:88-102, 2001.
Liaw et al., J. Biol. Chem., 275:5447-5452, 2000.
Marlar et al., Am. J. Hematol., 41:24-31, 1992.
Mazzone et al., Circulation, 88:358-363, 1993.
Muller et al., Eur. J. Pediatr., 155:20-25, 1996.
Murakami et al., Am. J. Physiol., 272:L197-L202, 1997.
Nakamura et al., In: Handbook of Experimental Immunology (4^{th} Ed.), Weir(Eds)., 1:27, Blackwell Scientific Publ., Oxford, 1987.
Napoli, J. Card. Surg., 17:355-362, 2002.
Oganesyan et al., J. Biol. Chem., 277:24851-24854, 2002.
Ozawa et al., Thromb. Res., 81:595-596, 1996.
Paret et al., J. Pediatr., 132:558, 1998.
PCT/US95/09636
Pepe et al., Thromb. Haemost., 77:329-331, 1997.
Petri, M., Lupus, 9:170-175, 2000.
Petrini et al., Pediatr. Hematol. Oncol, 7:165-175, 1990.
Regan et al., J. Biol. Chem., 271:17499-17503, 1996.
Reitsma et al., Thromb. Haemost., 73:876-889, 1995.
Riewald et al., Science, 296:1880-1882, 2002.
Rivard et al., J. Pediatr., 126:646-652, 1995.
Rodeghiero and Tosetto, Ann. Intern. Med., 130:643-650, 1999.
Rosendaal et al., Blood, 85:1504-1508, 1995.
Rosing et al., Lancet., 354:2036-2040, 1999.
Sakataetal., Blood, 68:1218-1223, 1986.
Sakkinen et al., J. Clin. Epidemiol., 55:445-451, 2002.
Salomaa et al., Atherosclerosis, 157:309-314, 2001.
Sanz-Rodriguez et al., Thromb. Haemost., 81:887-890, 1999.
Stearns-Kurosawa et al., Proc. Natl. Acad. Sci. USA, 93:10212-10216, 1996.
Steams-Kurosawa et al., Blood, 99:526-530, 2002.
Steams-Kurosawa et al., J. Thromb. Haemost., 1:855-856, 2003.
Takahashi et al., Am. J. Hematol., 41:32-39, 1992.
Takano et al., Blood, 76:2024-2029, 1990.
Taube et al., Blood, 93:3792-3797, 1999.
Taylor et al., J. Clin. Invest., 79:918-925, 1987.
Taylor et al., Blood, 95:1680-1686, 2000.
Taylor, Jr., Crit. Care Med., 29:S78-S89, 2001.
Urowitz et al., Am. J. Med., 69:221-225, 1976.
van der Bom et al., Ann. Intern. Med., 125:265-269, 1996.
van der Meer et al., Arch. Intern. Med., 153:1557-1562, 1993.
van der Wal et al., Circulation, 89:36-44, 1994.
Vita et al., Circulation, 81:491-497, 1990.
Wada et al., Am. J. Hematol., 39:20-24, 2000.
Wcisel et al., Chest, 121:292-295, 2002.
White et al., Br. J. Haematol., 110:130-134, 2000.
Xu et al., J. Bio. Chem., 275:6038-6044, 2000.
Zivelin et al., Blood, 94:895-901, 1999.

1. A method for predicting occurrence of an acute cardiovascular event in a subject comprising measuring circulating levels of soluble endothelial protein C receptor (sEPCR) in said subject, wherein elevated sEPCR levels, as compared to levels in normally distributed controls, predict occurrence of an acute cardiovascular event in said subject.

2. The method of claim 1, wherein the sEPCR is measured by an immunoassay.

3. The method of claim 2, wherein the sEPCR is measured by ELISA.

4. The method of claim 1, wherein the sEPCR level is determined by measuring sEPCR in a blood product, cerebrospinal fluid or urine.

5. The method of claim 4, wherein the blood product is plasma or serum.

6. The method of claim 1, wherein the acute cardiovascular event is selected from the group consisting of myocardial infarction, stroke, angina pectoris and sudden coronary death.

7. The method of claim 1, further comprising assessing another cardiovascular disease risk factor in said subject.

8. The method of claim 7, wherein said other cardiovascular disease risk factor may comprise increased age, male gender, dyslipidemias, hypertension, C-reactive protein, hyperhomocysteinemia, lipoprotein(a), fibrinogen, obesity, physical inactivity, tobacco use, oral contraceptives, underlying primary disease, and other hematological cardiovascular risk factors.

9. The method of claim 1, further comprising administering a cardiovascular disease prevention program to said subject.

10. The method of claim 9, wherein said cardiovascular disease prevention program is selected from the group consisting of diet and life style changes, low-dose aspirin, cholesterol reducing agents, blood pressure reducing agents, and oral anticoagulant treatment.

11. The method of claim 10, further comprising assessing sEPCR after administering said cardiovascular disease prevention program to said subject.

12. The method of claim 1, further comprising administering a cardiovascular disease therapeutic program to said subject.

13. The method of claim 12, wherein said cardiovascular disease therapeutic program is selected from the group consisting of beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists, statins, cytokine blockers/inhibitors and low fat diet.

14. The method of claim 12, further comprising assessing sEPCR after administering the cardiovascular disease therapeutic program to the subject.

15. The method of claim 1, wherein an elevated sEPCR is greater than about 150 ng/ml serum.

16. The method of claim 1, wherein said subject (a) does not suffer from cardiovascular disease; (b) suffers from previously undiagnosed cardiovascular disease; or (c) suffers from previously diagnosed cardiovascular disease.

17. A method for predicting or diagnosing atherosclerotic cardiovascular disease in a subject comprising measuring circulating levels of soluble endothelial protein C receptor (sEPCR) in said subject, wherein elevated sEPCR levels, as compared to levels in normally distributed controls, predict development or diagnose presence of atherosclerotic cardiovascular disease in said subject.

18. The method of claim 17, wherein the sEPCR is measured by an immunoassay.

19. The method of claim 18, wherein the sEPCR is measured by ELISA.

20. The method of claim 17, wherein the sEPCR level is determined by measuring sEPCR in a blood product, cerebrospinal fluid or urine.

21. The method of claim 20, wherein the blood product is plasma or serum.

22. The method of claim 17, wherein said subject is does not suffer from atherosclerotic cardiovascular disease.

23. The method of claim 22, further comprising administering a atherosclerotic cardiovascular disease prevention program to said subject.

24. The method of claim 23, wherein said cardiovascular disease prevention program is selected from the group consisting of low-dose aspirin, cholesterol reducing agents, blood pressure reducing agents and oral anticoagulant treatment.

25. The method of claim 23, further comprising assessing sEPCR after administering said cardiovascular disease prevention program to said subject.

26. The method of claim 17, wherein said subject has previously undiagnosed atherosclerotic cardiovascular disease.

27. The method of claim 26, further comprising administering a cardiovascular disease therapeutic program to said subject.

28. The method of claim 27, wherein said cardiovascular disease therapeutic program is selected from the group consisting of beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists, statins, cytokine blockers/inhibitors and low fat diet.

29. The method of claim 27, further comprising assessing sEPCR after administering said cardiovascular disease therapeutic program to said subject.

30. The method of claim 16, wherein an elevated sEPCR is greater than about 150 ng/ml serum.
